(19) United States
(12) Patent Application Publication
(10) Pub. No.: US 2016/0168541 A1
(43) Pub. Date: Jun. 16, 2016

(54) TRANSDUCIBLE MATERIALS FOR CELL REPROGRAMMING

(71) Applicant: VIVOSCRIPT, INC., Costa Mesa, CA (US)

(72) Inventors: Yong ZHU, Anaheim, CA (US); Huei-Sheng Vincent CHEN, La Jolla, CA (US); Shili WU, Edmond, OK (US); Jun BAO, Rowland Heights, CA (US)

(21) Appl. No.: 14/908,534

(22) PCT Filed: Jul. 29, 2014
(86) PCT No.: PCT/US14/48646

§ 371 Date: Jan. 28, 2016

Related U.S. Application Data
(60) Provisional application No. 61/958,493, filed on Jul. 29, 2013.

(51) Int. Cl. C12N 5/0775; C07K 14/47; C07K 14/435

(52) U.S. Cl. CPC C12N 5/0662; C07K 14/43595; C07K 14/47; C07K 2319/095; C07K 2319/09; C07K 2319/60; C12N 2506/13; C12N 2501/60; C12N 2501/999

(57) ABSTRACT

The present inventions are directed to compositions and methods regarding the reprogramming of biological samples (such as cells) without introducing exogenous genes to the sample. In particular, the present inventions are directed to transducible materials that are capable of transducing into the nuclei and have enhanced retention in the nuclei of the biological sample. The present inventions also are directed to methods of reprogramming a biological sample or treating diseases using the transducible compositions thereof.

### CROSS-REFERENCE TO RELATED APPLICATIONS

This application claims priority to U.S. provisional patent application No. 61/958,493, filed Jul. 29, 2013, the disclosure of which is incorporated herein by reference in their entirety.

### FIELD OF THE INVENTION

The present invention generally relates to tranducible materials for cell re-programming.

### BACKGROUND OF THE INVENTION

Cell fate reprogramming refers to a process in which a somatic cell de-differentiates into an induced pluripotent stem cell (iPSC) or trans-differentiates into a different type of somatic cell (the target cell). Cell fate reprogramming is of great clinical interest because the iPSCs or the target cells are derived from one's own somatic cells and can be used for tissue therapy without immunological rejection. A number of genetic approaches have been developed for cell fate reprogramming via viral transduction (see e.g., Takahashi and Yamanaka, Induction of pluripotent stem cells form mouse embryonic and adult fibroblast cultures by defined factors, Cell, 126(4), 663-676 (2006), and Stadtfeld et al., Induced pluripotent stem cells generated without viral integration, Science, 322, 945-949 (2008)), transient transfection (Okita et al., Generation of germline-competent induced pluripotent stem cells, Nature, 448 313-317 (2007)) or a transposition system (Woltjen et al., PiggyBac transposition reprograms fibroblasts to induced pluripotent stem cells, Nature, 458, 766-770 (2009)). However, the use of viral and other modified method all involve the use of genetic materials with drawbacks of genome modification by exogeneous sequences in target cells and having inadequate control of expression level of transgenes.

Cell fate reprogramming via reprogramming proteins possesses excellent safety and convenience for it does not involve any change of cellular genome. During the whole protein reprogramming processes, the main difficulty is how to transduce protein into nuclei and initiate reprogramming procedures. First, the protein must be transported across cytomembrane. A number of approaches have been developed to facilitate transportation across cell membrane, including fusing the protein with a supercharged protein or a cell penetrating peptide (see Zhou et al., Generation of induced pluripotent stem cells using recombinant proteins, Cell Stem Cell, 4, 381-384 (2009); Kim et al., Generation of human induced pluripotent stem cells by direct delivery of reprogramming proteins, Cell Stem Cell, 4 472-476 (2009)). However, once transported into the cells, most of such fusion proteins are trapped in the endosomes and cannot escape into the cytosol and further shuttled into the nuclei, resulting in low reprogramming efficiency. Therefore, there are needs in the field to develop new materials and methods to transduce reprogramming proteins into nuclear and increase the reprogramming efficiency.

### BRIEF SUMMARY OF THE INVENTION

In one aspect, the present disclosure relates to a tranducible material comprising an isolated polypeptide capable of transducing and retaining in a cell nucleus, wherein the isolated polypeptide comprises an effector domain. In certain embodiments, the effector domain comprises an amino acid sequence mutated from a nuclear export signal. In certain embodiments, the NES comprises an amino acid sequence of Y¹-X₍₂₋₃₎-Y²-X-Y³, wherein Y¹, Y² and Y³ are independently selected from the group consisting of leucine, isoleucine, valinine, phenylalanine and methionine, and X=spacing amino acid. In certain embodiments, the NES comprises an amino acid residue sequence of LX₍₁₋₃₎LX₍₂₋₃₎LXL, wherein L=leucine and X=spacing amino acid. In certain embodiments, the NES is mutated by replacing at least one of Y¹, Y² and Y³ in the NES with an alanine.

In certain embodiments, the effector domain is capable of exerting reprogramming changes of a biological sample once transduced into a biological sample. In certain embodiments, the effector domain is inherently capable of transducing into the biological sample.

In certain embodiments, the transducible material further comprises a nuclear localization signal, which is covalently or non-covalently associated with or linked to the effector domain. In certain embodiments, the NLS is exogenous to the effector domain. In certain embodiments, the NLS is covalently linked to the effector domain through a linker.

In certain embodiments, the transducible material further comprises a transduction domain, which is covalently or non-covalently associated with or linked to the effector domain. In certain embodiments, the tranduction domain is covalently linked to the effector domain through a linker.

In certain embodiments, the transducible material is capable of selectively transducing into one or more specific biological samples or becoming transducible in a specific environment surrounding the biological sample.

Another aspect of the present disclosure relates to a composition comprising a biological sample and a transducible material, wherein the transducible material has transduced into the biological sample.

Another aspect of the present disclosure relates to a method of reprogramming a biological sample by exposing the biological sample to a composition comprising a transducible material.

Another aspect of the present disclosure relates to a method of treating a disease or condition in a biological organism comprising administering a pharmaceutical composition comprising a transducible material into the biological organism.

Another aspect of the present disclosure relates to a method of treating a disease or condition in a biological organism comprising transplanting a biological sample into the biological organism, wherein the biological sample is transduced with a transducible material.

Another aspect of the present disclosure relates to a method for reprogramming a biological sample, comprising: exposing the biological sample to a tranducible material comprising an effector domain; and blocking the nuclear export of the transducible material.

Another aspect of the present disclosure relates to a method for identifying a transducible material comprising an isolated polypeptide capable of transducing and retaining in a cell nucleus, wherein the isolated polypeptide comprises an effector domain, the method comprising: modifying the isolated polypeptide; introducing the modified isolated polypeptide into a biological sample; and observing the localization of the modified isolated polypeptide in the biological sample.

### BRIEF DESCRIPTION OF THE FIGURES

FIG. 1. Schematic of a polypeptide comprising an effector domain with a mutated NES. A super charged GFP and a transduction domain of 11R are fused at the N-terminus and C-terminus of the effector domain respectively.

FIG. 2. Comparison of sGFP-Oct4(mNES) and sGFP-Oct4 protein in regulating the expression of downstream genes. HepG2 cells were treated with sGFP, sGFP-Oct4 or sGFP-Oct4(mNES) in serum-free medium for 6 hours. RNA was isolated and RT-qPCR was performed. The expression of each gene was normalized by the expression level of GAPDH. The relative expression level of each gene in the control condition (sGFP-treated sample) was set as 1.00.

FIG. 3. Schematic of the three generations of transducible materials derived from MyoD. (A) The first generation takes the form of sGFP-MyoD-11R. (B) The second generation has a classical NLS (SV40 T antigen NLS) inserted between sGFP and MyoD. (C) The third generation is the 2nd generation with the NES disrupted by site-directed mutagenesis.

FIG. 4. Schematic of experimental protocol to convert fibroblasts to myoblasts with transducible materials derived from MyoD.

FIG. 5. Live fluorescent imaging of human fibroblasts after culturing in 10 μg/ml of first generation sGFP-MyoD-11R. At 10 μg/ml, sGFP-MyoD-11R polypeptides entered the cells but were confined in the cytoplasmic compartment with minimal nuclear occupancy.

FIG. 6. The second generation of transducible materials derived from MyoD showed better nuclear stay than the first generation, but its nuclear occupancy is not sustainable. (A) Adult human fibroblasts were cultured with 10 μg/ml sGFP-NLS-MyoD-11R polypeptides, and GFP signal were observed in the nuclei. (B) The GFP signal in the nuclei decreased after 24 hours, indicating that most of the sGFP-NLS-MyoD-11R polypeptides were exported. (C) Adding leptomycin B (5 nM) slightly improves the nuclear occupancy of sGFP-NLS-MyoD-11R.

FIG. 7. The third generation of transducible material derived from MyoD significantly increased nuclear occupancy. Adult human fibroblasts were cultured with 10 μg/ml sGFP-NLS-MyoD(mNES)-11R polypeptides with 5 nM leptomycin B. Strong GFP signal was observed in nuclei at 30 min (FIG. 7A), 24 hours (FIG. 7B) and 48 hours (FIG. 7C) after culturing.

FIG. 8. Fibroblasts were induced to differentiate into satellite cells (SCs) and myoblasts by the third generation of transducible materials derived from MyoD. (A) At 18-22 D after exposure to sGFP-NLS-MyoD(mNES)-11R polypeptides and leptomycin B, human fibroblasts were converted to Pax7+/Myf5+ SCs. (B) After induction by myogenic media, SCs differentiated into endogenous MyoD+ myoblasts with positive demsin staining. No GFP signal from sGFP-NLS-MyoD(mNES)-11R polypeptide could be detected after 22 days after the polypeptide application.

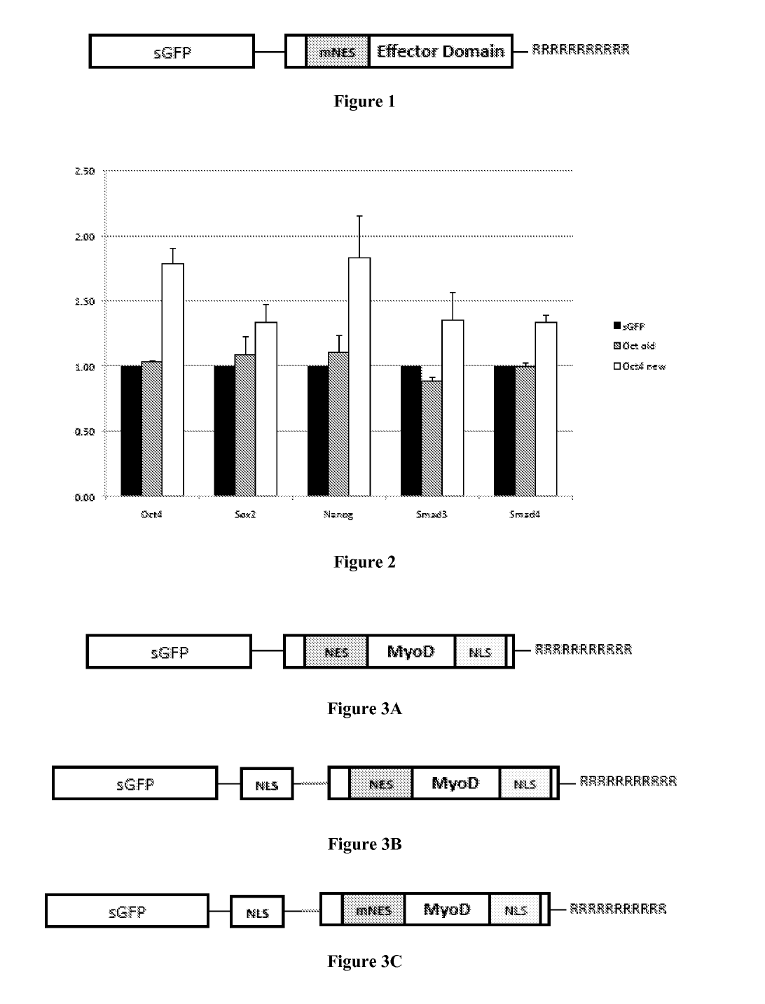

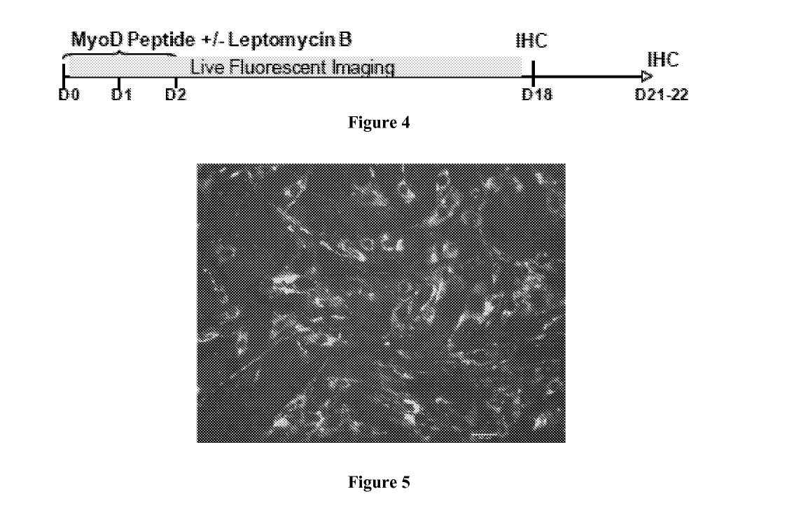

### DETAILED DESCRIPTION OF THE INVENTION

The present disclosure relates to a transducible material capable of being transduced into the nuclei of a biological sample. In particular, the transducible material has increased retention in the nuclei, and as such, is capable of reprogramming the biological sample in a higher efficiency.

The present disclosure is based on the discovery that in order to achieve a high efficiency of reprogramming, a transducible material must overcome three hurdles. First, the transducible material must be transported across cytomembrane. As the functional component of a transducible material that exerts reprogramming function is often protein-based, which by itself usually cannot across cytomembrane, the transducible material must be engineered to facilitate the functional component to be uptake by the biological sample; second, as most functional components are derived from transcription factors or other nuclear proteins, which must enter the nuclei to become functional, the functional component, after being uptake by the biological sample, must be translocated into the nuclei. The inventors of the present disclosure found that once transported into the cells, most of the transducible materials tend to be trapped in the endosomes and cannot escape into the cytosol and further shuttled into the nuclei, resulting in low reprogramming efficiency. Third, the inventors of the present disclosure found that after nuclear translocation, the transducible materials tend to be pumped back to cytoplasm via the nuclear exporting system in a very short period of time, resulting in low reprogramming efficiency. In certain embodiments, the inventors surprisingly found that retention of a transducible material in the nuclei of a biological sample for longer time is critical for reprogramming the biological sample, and enhanced nuclear retention of a transducible material significantly increased the reprogramming efficiency.

One aspect of the present disclosure relates to a tranducible material comprising an isolated polypeptide capable of being retained in a cell nucleus, wherein the isolated polypeptide comprises an effector domain.

In certain embodiments, a transducible material used herein refers to a molecule or a composition capable of crossing a membrane of a biological sample (e.g., a cell membrane) so that the transducible material can enter or be brought into the inside of the biological sample from the outside of the biological sample and exerts reprogramming effects. For example, the transducible material may interact with cell-surface receptors which facilitate the entry of the material into cells through receptor mediated endocytosis.

In certain embodiments, a transducible material is selectively tranducible, i.e., the transducible material is more likely to transduce into a specific type of biological samples (e.g., cancer or tumor cells) or becomes transducible in a specific microenvironment in or around a biological sample (e.g., microenvironment around cancer or tumor) than other biological samples. For example, the selective transducible material comprises a transducible domain (e.g., a cell-targeting peptide or an activatable cell penetrating peptide) that preferably delivers the selective transducible material into a specific type of biological sample or become transducible in a microenvironment around a biological sample.

Without being bounded to any theories, it is contemplated that the transducible materials may cross a cell membrane and enter into cytoplasm to reprogram cytoplasm activities such as translation, post translation modification, signaling pathway, apoptosis pathway. It is further contemplated that the transducible material may cross the nucleus membrane and reprogram or modulate DNA or chromosomal replication, gene transcription or RNA splicing.

The term “polypeptide” as used herein refers to a compound made up of a single chain of amino acid residues linked by peptide bonds. The term “protein” as used herein is used interchangeably with the term “polypeptide”.

The term “isolated” as used herein refer to a polypeptide that is removed from at least one component with which it is naturally associated.

The term “retain” or “retention” in nuclei as used herein refers to the nuclear occupancy of the isolated polypeptide. In certain embodiments, retention in nuclei means that the isolated polypeptide stays in nuclei for more than 24 hours. In certain embodiments, retention in nuclei means that the isolated polypeptide stays in nuclei for more than 48 hours. In certain embodiments, retention in nuclei means that the isolated polypeptide stays in nuclei for more than 72 hours.

In certain embodiments, the isolated polypeptide comprises an effector domain, wherein the effector domain comprises an amino acid sequence mutated from a nuclear export signal (NES).

An effector domain as used herein is a polypeptide or a fragment thereof that, once inside a biological sample, is capable of exerting reprogramming changes of the biological sample. The effector domain may interact with molecules (e.g., proteins, DNA, RNA, sugars, and lipids) in the biological sample (e.g., in cytoplasm or nuclei) and lead to changes such as proliferation, differentiation, de-differentiation, transdifferentiation, retro-differentiation, transdetermination, apoptosis, and morphogenesis.

In certain embodiments, an effector domain is a nuclear protein such as a transcription factor, a chromosome remodeling protein and enzymes involved in DNA replication, transcription and repair, or a fragment or mimic thereof.

In certain embodiments, the effector domain comprises an amino acid sequence mutated from a nuclear export signal (NES). A NES is a short amino acid sequence in a protein that targets it for export from the cell nucleus to the cytoplasm. This export is mostly mediated by the karyopherin exportin 1/chromosomal region maintenance 1 (CRM1) recognizing the NES of the cargo molecules. The classical NES is characterized by three to four conserved hydrophobic residues, usually leucine, and the spacing between them. A common classical NES has the consensus sequences of LX₍₁₋₃₎LX₍₂₋₃₎LXL, where L=Leu, X=spacing amino acid (i.e., any amino acid residue found in a polypeptide including alanine, arginine, asparagine, aspartic acid, cysteine, glutamic acid, glutamine, glycine, histidine, isoleucine, leucine, lysine, methionine, phenyalanine, proline, serine, threonine, tryptophan, tyrosine and valine). More recently, a consensus sequence of NES of Y¹-X₍₂₋₃₎-Y²-X-Y³, wherein Y¹, Y² and Y³ are independently selected from the group consisting of leucine, isoleucine, valinine, phenylalanine and methionine, and X=spacing amino acid, has been proposed (Fu et al., Prediction of leucine-rich nuclear export signal containing proteins with NESsential, Nucleic Acids Res., 39:e111 (2011)). Some examples of NES are listed in Table 1. In addition, several database has been established with collection of NES-containing proteins or for NES prediction, including NESbase, NetNES and ValidNESs.

TABLE 1
Examples of NES.

| Y¹-XX-Y²-X-Y³ | Y¹-XXX-Y²-X-Y³ |
|---|---|
| LXXXLXXLXL | LXXXLXXXLXL |
| LXXLXXXLXL | LXXLXXLXL |
| LXLXXLXL | LXLXXXLXL |

Y¹, Y² and Y³ are independently selected from the group consisting of leucine, isoleucine, valinine, phenylalanine and methionine, L = leucine and X = spacing amino acid

The term “mutate” or “mutated” as used herein refers to a change of the amino acid sequence of a NES such that the function of the NES to export the protein out of cell nuclei is disrupted. As a result, the retention of the effector domain within nuclei will be enhanced. In certain embodiments, enhanced retention in the nucleus increases the half-life of the transducible material in a biological sample. In certain embodiments, enhanced retention of a protein in the nucleus shift the distribution of the protein in the cell so that the protein escapes from the endosome and further enters the cell nuclei.

In certain embodiments, a NES is mutated by replacing the hydrophobic residues in the NES sequence (e.g., leucine, isoleucine, valinine, phenylalanine or methionine) with alanine. In certain embodiments, a NES is mutated by replacing at least one hydrophobic residue with alanine. In certain embodiments, a NES is disrupted by replacing at least two hydrophobic residues with alanine. In certain embodiments, a NES is disrupted by replacing at least three hydrophobic residues with alanine. In certain embodiments, a NES is disrupted by replacing at least four conserved hydrophobic residues with alanine. In certain examples, the hydrophobic residues being replaced in the NES is leucine. For example, a NES with consensus sequence of LX₍₁₋₃₎LX₍₂₋₃₎LXL can be mutated to AX₍₁₋₃₎LX₍₂₋₃₎LXL, LX₍₁₋₃₎AX₍₂₋₃₎LXL, LX₍₁₋₃₎LX₍₂₋₃₎AXL, LX₍₁₋₃₎LX₍₂₋₃₎LXA, AX₍₁₋₃₎AX₍₂₋₃₎LXL, AX₍₁₋₃₎LX₍₂₋₃₎AXL, AX₍₁₋₃₎LX₍₂₋₃₎LXA, LX₍₁₋₃₎AX₍₂₋₃₎AXL, LX₍₁₋₃₎AX₍₂₋₃₎LXA, LX₍₁₋₃₎LX₍₂₋₃₎AXA, AX₍₁₋₃₎AX₍₂₋₃₎AXL, AX₍₁₋₃₎AX₍₂₋₃₎LXA, AX₍₁₋₃₎LX₍₂₋₃₎AXA or AX₍₁₋₃₎AX₍₂₋₃₎AXA. A NES with consensus sequences of Y¹-X₍₂₋₃₎-Y²-X-Y³ can be mutated to A-X₍₂₋₃₎-Y²-X-Y³, Y¹-X₍₂₋₃₎-A-X-Y³, Y¹-X₍₂₋₃₎-Y²-X-A, A-X₍₂₋₃₎-A-X-Y³, A-X₍₂₋₃₎-Y²-X-A, Y¹-X₍₂₋₃₎-A-X-A or A-X₍₂₋₃₎-A-X-A.

In certain embodiments, an effector domain is derived from a nuclear protein selected from the group consisting of Ascl1 (SEQ ID NO: 1), Baf60c (SEQ ID NO: 2), Brn2 (SEQ ID NO: 3), Brn4 (SEQ ID NO: 4), Chd5 (SEQ ID NO: 5), c-Myc (SEQ ID NO: 6), Dlx1 (SEQ ID NO: 7), Dlx2 (SEQ ID NO: 8), Foxp1 (SEQ ID NO: 9), Foxp2 (SEQ ID NO: 10), Foxp3 (SEQ ID NO: 11), Gata4 (SEQ ID NO: 12), Hand1 (SEQ ID NO: 13), Hes1 (SEQ ID NO: 14), Hes5 (SEQ ID NO: 15), Isl1 (SEQ ID NO: 16), Lhx2 (SEQ ID NO: 17), Klf4 (SEQ ID NO: 18), MafA (SEQ ID NO: 19), Mef2c (SEQ ID NO: 20), Mesp1 (SEQ ID NO: 21), MyoD (SEQ ID NO: 22), Myt1L (SEQ ID NO: 23), Nanog (SEQ ID NO: 24), Nap1L2 (SEQ ID NO: 25), NeuroD1 (SEQ ID NO: 26), NeuroD2 (SEQ ID NO: 27), Ngn2 (SEQ ID NO: 28), Ngn3 (SEQ ID NO: 29), Nhlh2 (SEQ ID NO: 30), Nkx2.2 (SEQ ID NO: 31), Nkx2.5 (SEQ ID NO: 32), Nkx6.1 (SEQ ID NO: 33), Npas4 (SEQ ID NO: 34), Nrip3 (SEQ ID NO: 35), Oct4 (SEQ ID NO: 36), Oligo2 (SEQ ID NO: 37), Pax6 (SEQ ID NO: 38), Pax7 (SEQ ID NO: 39), Pdx1 (SEQ ID NO: 40), Prdm8 (SEQ ID NO: 41), Satb2 (SEQ ID NO: 42), Shox2 (SEQ ID NO: 43), Sox2 (SEQ ID NO: 44), Sox9 (SEQ ID NO: 45), Tbr1 (SEQ ID NO: 46), Tbx5 (SEQ ID NO: 47), Tbx18 (SEQ ID NO: 48), Zic1 (SEQ ID NO: 49).

In certain embodiments, the term “derive” or “derived” as used herein refers to a polypeptide comprising an amino acid sequence substantially identical to the sequence of a protein (e.g., a nuclear protein) or a fragment thereof that contains an innate NES, except that the amino acid sequence of the polypeptide that corresponding to the NES is mutated so that the function of the NES is disrupted.

In certain embodiments, an effector domain comprises an amino acid sequence selected from the group consisting of SEQ ID NO: 50-98.

In certain embodiments, the transducible material further comprises an agent that inhibits nuclear export. In certain embodiments, the agent is leptomycin B.

In certain embodiments, the transducible material further comprises a nuclear localization signal (NLS). The NLS is associated with the effector domain covalently, noncovalently or via a linker. In certain embodiments, the NLS is covalently linked to the effector domain through a linker. In certain embodiments, the linker is a glycine-rich linker that comprises one or more glycine residues (e.g. ESGGGGSPG (SEQ ID NO: 99), GGSGGSGGSGGSH (SEQ ID NO: 100)).

NLS are short peptide motifs that mediate the nuclear import of proteins by binding to their receptors, known as importins (karyopherins). Typically, an NLS consists of one or more short sequences of positively charged lysine's or arginines exposed on the protein surface. An NLS has the opposite function of a NES, which targets proteins out of the nucleus. NLSs mediated by the importins pathway has been well characterized (see Kosugi et al., Six classes of nuclear localization signals specific to different binding grooves of importin α, Journal of Biol. Chemistry, 284, 478-485 (2008)). In general, NLSs can be grouped into classical NLSs, whose consensus sequence has been defined, and non-classical NLSs. Classical NLSs can be further classified as either monopartite or bipartite. Monopartite NLSs have a single cluster of three or more consecutive basic amino acid residues. Examples of monopartite NLSs include the SV40 large T antigen NLS (PKKKRKV (SEQ ID NO: 101)) and the c-Myc NLS (PAAKRVKLD (SEQ ID NO: 102)). Bipartite NLSs have two clusters of basic amino acids separated by a 10-12-amino acid linker, and have a consensus sequence of (K/R)(K/R)X₁₀₋₁₂(K/R)_{3/5}, where (K/R)_{3/5} represents at least three of either lysine or arginine of five consecutive amino acids. An example of bipartite NLS is the nucleoplasmin NLS (KRPAATKKAGQAKKKK (SEQ ID NO: 103)). Examples of non-classical NLSs include acidic M9 domain of hnRNP A1, the sequence KIPIK in yeast transcription repressor Matα2, and the complex signals of U snRNPs.

In certain embodiments, an effector domain contains an innate NLS. In certain embodiments, the NLS is exogenous to the effector domain, which means the NLS associated with the effector domain is not an innate NLS of the effector domain.

In certain embodiments, associating a NLS with an effector domain is capable of facilitating the entry of the transducible material into the nucleus of a biological sample. In certain embodiments, associating a NLS with an effector domain is capable of shifting the distribution equilibrium of the transducible material among different intracellular compartments towards nuclear localization. In certain embodiments, associating a NLS with an effector domain is capable of facilitating the transducible material escaping from the endosome of a biological sample.

In certain embodiments, a transducible material further comprises a transduction domain. A transduction domain is a motif that is capable of facilitating the entry of the transducible material into a biological sample (e.g., a cell). The transducible domain is associated with the effector domain covalently, noncovalently or via a linker. In certain embodiments, the transduction domain is covalently linked to the effector domain through a linker. In certain embodiments, the linker is a glycine-rich linker that comprises one or more glycine residues.

Examples of a transduction domain include, without limitation, polymers such as cationic lipid polymers and nanoparticles, protein transduction domains (PTD), cell penetrating peptides (CPP1), cell permeating peptides (CPP2), activatable cell penetrating peptides or conjugates (ACPP), and cell-targeting peptides (CTP).

Examples of a transduction domain has been disclosed in PCT Application PCT/US2009/069518, published as WO2010075575, which is incorporated herein by reference in their entirety.

In certain embodiments, a transducible domain is a protein transduction domain selected from the group consisting of super charged protein, TAT, poly-arginine, Penetratin, Antennapedia, VP22, Transportan, MAP, MTS, PEP-1, Arg/Trp analogue, RRWRRWWRRWWRRW, polyguanidine peptoid, polyguanidine peptoid, inherent protein transduction domain, SEQ ID NO: 104, HIV-1 Rev, Flock house virus coat peptide, DNA-binding peptides, c-Fos, c-Jun, yeast GCN4, and Fusogenic HA2 peptide. In one example, a transducible domain is a supercharged green fluorescent protein (sGFP) (SEQ ID NO: 105).

In certain embodiments, a transducible material comprises an effector domain that is inherently transducible, and a transduction domain associated with the effector domain via covalent or non-covalent interactions.

In certain embodiments, a transducible material further comprises one or more motifs that do not interrupt the function of the effector domain or the transduction domain. In certain embodiments, these motifs are linked covalently, non-covalently or through a linker to the effector domain and/or the transduction domain. In certain embodiments, these motifs facilitate the preparation and/or purification of the transducible material. One example of such motif is a polyhistidine-tag to facilitate protein purification in preparation of the transducible material. In certain embodiments, the polyhistidine-tag comprises at least six histidine residues (e.g. MGSSHHHHHHSSGLVPRGSH (“His6,” SEQ ID NO: 106)).

In certain embodiments, a tranducible material comprises an isolated polypeptide that has the form of TD-linker-NLS-linker-ED(mNES), where TD=transduction domain, ED(mNES)=effector domain with a disrupted NES. In certain embodiments, a transduction material comprises an isolated polypeptide that has the form of TD-linker-ED(mNES)-linker-NLS. In certain embodiments, a transduction material comprises an isolated polypeptide that has the form of NLS-linker-TD-linker-ED(mNES). In certain embodiments, a transduction material comprises an isolated polypeptide that has the form of TD-linker-NLS-linker-ED(mNES).). In certain embodiments, a transduction material comprises an isolated polypeptide that has the form of TD1-linker-NLS-linker-ED(mNES)-linker-TD2. In certain embodiments, a tranducible material comprising an isolated polypeptide that has the form of sGFP-linker-ED(mNES)-11R. In certain embodiments, a tranducible material comprising an isolated polypeptide that has the form of sGFP-linker-NLS-linker-ED(mNES)-11R. In certain embodiments, a transducible material comprises an isolated polypeptide that comprises an amino acid sequence selected from the group consisting of SEQ ID NO: 107-155.

Another aspect of the present disclosure relates to a composition comprising a biological sample and at least one transducible material, wherein the transducible material has transduced into the biological sample. For example, the composition includes a transducible material comprising an isolated polypeptide derived from Foxp3 (e.g. sGFP-Foxp3(mNES)-11R) and a T cell wherein the transducible material has transduced into the T cell. For another example, a composition includes a iPS cell and one or more transducible materials comprising an isolated polypeptide derived from a protein selected from the group consisting of Oct4, Klf4, Sox2 and c-Myc, and any combination thereof (e.g. sGFP-Oct4(mNES)-11R, sGFP-Klf4(mNES)-11R, sGFP-Sox2(mNES)-11R, sGFP-c-Myc(mNES)-11R). In yet another example, a composition including a liver or pancreatic exocrine cell and one or more transducible materials comprising a polypeptide derived from a protein selected from the group consisting of Ngn3, Pdx1, MafA, NeuroD1, and any combination thereof (e.g. sGFP-Ngn3(mNES)-11R, sGFP-Pdx1(mNES)-11R, sGFP-MafA(mNES)-11R, sGFP-NeuroD1(mNES)-11R) wherein the transducible materials have transduced into the liver or pancreatic exocrine cell. In yet another example, a composition including a fibroblast cell and a transducible material comprising a polypeptide derived from MyoD (e.g., sGFP-MyoD(mNES)-11R, sGFP-NLS-MyoD(mNES)-11R), wherein the transducible material has transduced into the fibroblast cell.

Another aspect of the present disclosure relates to a method of reprogramming a biological sample by exposing the biological sample to a composition comprising a transducible material. In certain embodiments, the method preferably reprograms a specific type of biological sample (e.g. cancer or tumor cells) or biological samples in or around a specific microenvironment within a biological organism (e.g. microenvironment around cancer or tumor) than other biological samples by exposing biological samples to a composition comprising a selective transducible material.

In one embodiment, a biological sample includes a cell, a cluster of cells, a tissue, an organ, a biological body from a biological organism. The biological sample can be normal, healthy sample or abnormal, diseased sample (e.g., cancer or tumor).

A biological organism includes, for example, a microorganism (e.g., bacteria), a fungus, a plant and an animal (e.g., a human).

An organ from an animal biological organism (e.g., human) includes, for example, a circulatory organ (e.g., heart, blood and blood vessels), a digestive organ (e.g., salivary glands, esophagus, stomach, liver, gallbladder, pancreas, intestines, rectum and anus), an endocrine organ (e.g., endocrine glands such as the hypothalamus, pituitary or pituitary gland, pineal body or pineal gland, thyroid, parathyroids and adrenals, i.e., adrenal glands), an integumentary organ (e.g., skin, hair and nails), a lymphatic organ (e.g., lymph nodes and vessels, tonsils, adenoids, thymus and spleen), a muscular organ (e.g., muscles), a nervous organ (e.g., brain, spinal cord, peripheral nerves and nerves), a reproductive organ (e.g., ovaries, fallopian tubes, uterus, vagina, mammary glands, testes, vas deferens, seminal vesicles, prostate and penis), a respiratory organ (e.g., the pharynx, larynx, trachea, bronchi, lungs and diaphragm), a skeletal organ (e.g., bones, cartilage, ligaments and tendons), a urinary system (e.g., kidneys, ureters, bladder and urethra). An organ can be normal or healthy, and alternatively, abnormal or unhealthy (e.g., cancerous).

An organ from a plant biological organism includes, for example, root, stem, leaf, flower, seed and fruit.

A tissue from a biological sample (e.g. an animal) includes a connective tissue, a muscle tissue, a nervous tissue, and an epithelial tissue. A tissue can be normal or healthy, and alternatively, abnormal or unhealthy (e.g., cancerous). A tissue from a biological sample (e.g. a plant) includes an epidermis, a vascular tissue and a ground tissue.

A cell can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell).

A cell further includes a mammalian stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and an unipotent stem cell (See, Hans R. Schóler (2007). “The Potential of Stem Cells: An Inventory” in Nikolaus Knoepffler, Dagmar Schipanski, and Stefan Lorenz Sorgner. Humanbiotechnology as Social Challenge. Ashgate Publishing, Ltd. pp. 28), any of which may be induced from a somatic cell. A stem cell may also include a cancer stem cell.

In another embodiment, “reprogramming a biological sample” used herein is exchangeable with or refers to modulating, altering, or changing the biological activities of the biological sample (e.g., cell) or modulating, altering, or changing the state or status of the biological sample from one to another. For example, by exposing a biological sample (e.g., a cell) to a transducible material, the biological activities of the cell (e.g., cell growth, cell division, cell metabolism, cell cycle, cell signaling, DNA replication, transcription, RNA splicing, protein synthesis, post-translation modification) are modulated or altered so as to lead to cell proliferation, differentiation (e.g., from progenitor cells to terminally differentiated cells), dedifferentiation (e.g., from terminally differentiated cells to pluripotent stem cells), transdifferentiation (e.g., from one type of terminally differentiated cells to another type of terminally differentiated cells), retro differentiation (e.g., from terminally differentiated cells to progenitor cells), transdertermination (e.g., from one type of progenitor cells to a type of terminally differentiated cells that are usually derived from another type of progenitor cells under natural conditions), apoptosis (e.g., cell death of cells or cancer cells), morphogenesis, and changes in the cell fate. For another example, the state of a biological sample can be altered or changed from abnormal or diseased state to normal or healthy state (e.g., from cancer cells to noncancer cells); from one cell type to another cell type (e.g., from undifferentiated stem cells to differentiated stem cells or specialized cells), from differentiated or specialized cells to undifferentiated cells or stem cells (e.g., an omnipotent stem cell, a pluripotent stem cell, a multipotent stem cell, an oligopotent stem cell and an unipotent stem cell) (e.g., from fibroblast cells to induced pluripotent stem cells (iPSCs)), from somatic cells to stem cells or induced stem cells, from one state of stem cells to another state of stem cells (e.g., from ominipotent stem cells to pluripotent stem cells), from one type of differentiated cells to another type of differentiated cells (e.g., T-cells to regulatory T cells, pancreatic exocrine cells to insulin-producing beta cells).

In another embodiment, a biological sample is exposed to a transducible material and reprogrammed. The biological sample can be exposed in vitro, in vivo or ex vivo. For example, the biological sample is exposed in vitro through contacting the sample with the transducible material in an environment outside of a living biological organism (e.g., in a cell culture system or a test tube). The biological sample is exposed in vivo through contacting the material with a biological organism containing the sample or introducing (e.g., through administration) the material into the organism. The transducible materials can be administered via any known administration route such as for example parenteral {e.g., subcutaneous, intraperitoneal, intravenous, including intravenous infusion, intramuscular, or intradermal injection) or non-parenteral {e.g., oral, intranasal, intraocular, sublingual, rectal, or topical) route. The biological sample is exposed ex vivo when the biological sample (e.g., a cell, a tissue or an organ) is taken outside the biological organism, contacted with the transducible material, and placed back to the same or different biological organisms. Examples of ex vivo exposures comprise removing a biological sample from the biological organism, exposing the biological sample to a transducible material, and transplanting the biological sample transduced with the transducible material back to the biological organism.

In certain embodiments, OG2-MEF cells are exposed to a composition comprising protein sGFP-Ngn3(mNES)-11R, sGFP-Pdx1(mNES)-11R, sGFP-MafA(mNES)-11R and reprogrammed to induced pluripotent stem cells (iPSCs).

In certain embodiments liver and/or pancreatic exocrine cells are exposed to a composition comprising one or more proteins selected from the group consisting of sGFP-Ngn3(mNES)-11R, sGFP-Pdx1(mNES)-11R, sGFP-MafA(mNES)-11R and sGFP-NeuroD1(mNES)-11R, and reprogrammed into insulin producing cells (e.g. β cells). In certain embodiments, the composition further comprises one or more adjuvant such as Islet growth factor (e.g. betacellulin). Without bond to the mechanism, it is further contemplated that such reprogramming is through transdetermination and/or transdifferentiation.

In certain embodiments, adult fibroblast cells are exposed to a composition comprising sGFP-NLS-MyoD(mNES)-11R and reprogrammed into satellite cells.

Another aspect of the present disclosure relates to a method of treating, preventing or reducing a disease or condition in a biological organism by administering a composition comprising a transducible material into the organism. In certain embodiments, the composition is a pharmaceutical composition comprising a transducible material. In certain embodiments, the composition comprises a selective transducible material. The treatment, prevention or reduction of a disease or condition is associated with the change or reprogramming of a biological sample (e.g., a cell, a tissue or an organ) in the organism.

In certain embodiments, the disease or condition treatable by the method include, without limitations, tumor, cancer, metabolic diseases or conditions (e.g. type I and type II diabetes and obesity), inflammatory conditions, cardiac diseases, neuro generative diseases (e.g. anemia, amyotrophic lateral sclerosis, spinal cord injury, burns, or arthritis), autoimmune diseases or conditions (e.g. acute disseminated encephalomyelitis (ADEM), Addison's disease, alopecia areata, ankylosing spondylitis, antiphospholipid antibody syndrome (APS), anemia (e.g. autoimmune hemolytic anemia and pernicious anaemia), arthritis, psoriatic arthritis, rheumatoid arthritis, diabetes mellitus type 1, autoimmune hepatitis, autoimmune inner ear disease, bullous pemphigoid, coeliac disease, Chagas disease, chronic obstructive pulmonary disease, Crohns disease, dermatomyositis, endometriosis, Goodpasture's syndrome, Graves' disease, Guillain-Barre syndrome (GBS), Hashimoto's disease, hidradenitis suppurativa, Kawasaki disease, IgA nephropathy, idiopathic thrombocytopenic purpura, interstitial cyctitis, lupus erythematosus, mixed connective tissue disease, morphea, multiple sclerosis (MS), myasthenia gravis, narcolepsy, neuromyotonia, pemphigus vulgaris, psoriasis, polymyositis, primary billiary cirrhosis, schizophrenia, scleroderma, Sjogren's syndrome, stiff person syndrome, temporal arteritis (“Giant cell arteritis”), ulcerative colitis, vasculitis, vitiligo, and Wegener's granulomatosis).

For example, it is contemplated that a transducible material can be administered to a biological organism having a tumor to activate the apoptosis of the tumor cells or make tumor cells more sensitive to chemotherapy, radiotherapy, or cancer drugs.

In certain embodiments, a transducible material can be administered to a biological organism to treat metabolic diseases or conditions such as type I diabetes, type II diabetes, or obesity. For example, to treat diabetes, a composition comprising a protein selected from the group consisting of sGFP-Ngn3(mNES)-11R, sGFP-Pdx1(mNES)-11R, sGFP-MafA(mNES)-11R, sGFP-NeuroD(mNES)-11R and any combination thereof can be transduced into liver and/or pancreatic exocrine cells and programs them to insulin producing cells (e.g. β cells). In certain embodiments, one or more adjuvant such as Islet growth factor (e.g. betacellulin) is/are also administered to the biological organism. Without bond to the mechanism, it is further contemplated that such reprogramming is through transdetermination and/or transdifferentiation.

It is further contemplated that a transducible material can be administered to a biological organism to treat cardiac diseases such as myocardial infarction or ischemia.

Another aspect of the present disclosure relates to a method of reprogramming iPSCs, embryonic stem cells, or other types of stem or progenitor cells to certain types of somatic cells or progenitor cells, which can be developed as cell-based therapies for various diseases or conditions, including anemia, neurodegenerative diseases, cancer, amyotrophic lateral sclerosis, spinal cord injury, burns, heart diseases, diabetes, and arthritis. The stem cells or progenitor cells may be patient-specific or non-patient-specific, repaired to rid of molecular defects or not, before they are exposed to transducible materials for controlled differentiation or reprogramming. The reprogrammed cells may be enriched, purified, or manipulated before transplanted back to patients.

Another aspect of the present disclosure relates to a method of reprogramming iPSCs, embryonic stem cells, or other types of stem or progenitor cells to certain types of somatic cells or progenitor cells, which can be used as disease models for drug screening, mechanism study, toxicity assay, or other research and drug discovery and development tools. For example, the method comprises exposing an iPSC, an embryonic stem cell, or a progenitor cell to a composition comprising a transducible material to reprogram the iPSC, embryonic stem cell, or progenitor cell to a transplantable somatic cell or a transplantable progenitor cell; transplanting the transplantable somatic cell or transplantable progenitor cell into a biological sample or a biological organism; developing the biological sample or biological organism to become a disease model. For another example, the method comprises reprogramming patient-specific cells to iPSCs using a transducible materials; further generating different type of cells from patient specific iPSCs with or without tranducible materials; and developing a disease model using patient-specific iPSCs or iPSC-derived cells. For another example, the method of developing drug screening or toxicity models comprises reprogramming somatic cells, progenitor cells, or multipotent cells to iPSCs using a transducible material; further generating different type of cells from iPSCs with or without exposing to transducible materials; and using iPSCs and/or iPSC-derived cells to screen the effects and/or toxicities of different compounds.

Another aspect of the present disclosure relates to a method of developing cell-based therapies for various diseases or conditions comprising the step of reprogramming an iPSC, an embryonic stem cell, or a progenitor cell to a transplantable somatic or progenitor cell using a transducible material; transplanting the transplantable somatic or progenitor cell into a biological sample or biological organism; assessing the therapeutic effect of the transplantable somatic or progenitor cell.

In another aspect, the present disclosure relates to a method for reprogramming a biological sample, comprising: exposing the biological sample to a tranducible material comprising an effector domain; and blocking the nuclear export of the transducible material. In certain embodiments, the nuclear export of the transducible material is blocked by mutated the NES of the effector domain. In certain embodiments, the nuclear export of the transducible material is blocked by exposing the biological sample to an agent that is capable of inhibiting nuclear export.

In yet another aspect, the present disclosure relates to a method for identifying a transducible material comprising an isolated polypeptide capable of transducing and retaining in a cell nucleus, wherein the isolated polypeptide comprises an effector domain, the method comprising: modifying the isolated polypeptide; introducing the modified isolated polypeptide into a biological sample; and observing the localization of the modified isolated polypeptide in the biological sample.

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### EXAMPLE 1

The following is an example of identification of NES in a protein.

A NES is a short amino acid sequence in a protein that targets it for export from the cell nucleus to the cytoplasm. The classical NES is characterized by three to four conserved hydrophobic residues, usually leucine, and the spacing between them. A common classical NES has the consensus sequences of LX₍₁₋₃₎LX₍₂₋₃₎LXL, where L=Leu, X=spacing amino acid (i.e., any amino acid residue found in a polypeptide including alanine, arginine, asparagine, aspartic acid, cysteine, glutamic acid, glutamine, glycine, histidine, isoleucine, leucine, lysine, methionine, phenyalanine, proline, serine, threonine, tryptophan, tyrosine and valine). More recently, a consensus sequence of NES of [LIVFM-X₍₂₋₃₎-[LIVFM]-X-[LIVFM], where [LIVFM]=leucine, isoleucine, valinine, phenylalanine or methionine, X=spacing amino acid, has bee proposed (Fu et al., Prediction of leucine-rich nuclear export signal containing proteins with NESsential, Nucleic Acids Res., 39:e111 (2011)). Several databases has been established with collection of NES-containing proteins or for NES prediction, including NESbase, NetNES and ValidNESs.

Using NESbase, NetNES, ValidNESs or literature search, a number of nuclear proteins that comprises a NES were identified. (Table 2). In particular, using NetNES and similar tools, the presence of a NES in a protein can be predicted. The validation of the predicted NES can be achieved by methods known in the art. For example, a transgene can be prepared by fusing a reporter gene (e.g., GFP) to the gene of interest. The transgene can be expressed in a cell to observe the localization of the protein encoded by the gene of interest. The transgene can be mutated to replace the conserved hydrophobic amino acid residue (e.g., leucine) with alanine. The mutated transgene can then be expressed in a cell, and the localization of the mutated protein can be observed to determine whether the hydrophobic amino acid belong to a NES.

TABLE 2
Identification of NES
in nuclear proteins.

| Protein | SEQ | | | |
|---|---|---|---|---|
| Name | ID NO: | NES | | |
| Ascl1 | 1 | 167 | IRALQQLL | 174 |
| Baf60c | 2 | 268 | LARLLGL | 275 |
| Brn2 | 3 | 387 | LADSLQL | 393 |
| Brn4 | 4 | 311 | LADSLQL | 317 |
| Chd5 | 5 | 1648 | LDKLELSLI | 1656 |
| c-Myc | 6 | 442 | LKHKLEQL | 449 |
| Dlx1 | 7 | 161 | LASSLGL | 167 |
| Dlx2 | 8 | 185 | LAASLGL | 191 |
| Foxp1 | 9 | 62 | LQVARQLLL | 70 |
| Foxp2 | 10 | 44 | VSTVELLHL | 52, |
| | | 58 | LQAARQLLL | 66 |
| Foxp3 | 11 | 265 | LEQQLVLEKEKL | 276 |
| Gata4 | 12 | 44 | VPSSVLGLSYL | 54 |
| Hand1 | 13 | 130 | LSKIKTLRL | 138 |
| Hes1 | 14 | 54 | LSQLKTLIL | 62 |
| Hes5 | 15 | 36 | IEQLKLLL | 43 |
| Isl1 | 16 | 293 | VLSDFAL | 299 |
| Lhx2 | 17 | 75 | MRCLKCCECKLNL | 87 |
| Klf4 | 18 | 102 | LLDLDFIL | 109 |
| MafA | 19 | 293 | VEGLKL | 298 |
| Mef2c | 20 | 35 | LSVLCDCEIALII | 47 |
| Mesp1 | 21 | 123 | IETLRLAI | 130, |
| | | 133 | IGHLSAVLGL | 142 |
| MyoD | 22 | 43 | LLSPPLRDVDL | 13 |
| Myt1L | 23 | 249 | LSLDL | 253 |
| Nanog | 24 | 125 | MQELSNILNL | 134 |
| Nap1L2 | 25 | 268 | LLTDIKV | 274 |
| NeuroD1 | 26 | 137 | LSKIETLRL | 145 |
| NeuroD2 | 27 | 157 | LSKIETLRL | 165 |
| Ngn2 | 28 | 8 | LELKEEEDVLVLL | 20 |
| Ngn3 | 39 | 135 | LTQTLRI | 141 |
| Nhlh2 | 30 | 113 | LSKIEIL | 119 |
| Nkx2.2 | 31 | 53 | LDAVGSL | 59 |
| Nkx2.5 | 32 | 171 | LASVLKL | 177 |
| Nkx6.1 | 33 | 16 | LLSSPPLAALHSMAE | 30 |
| Npas4 | 34 | 35 | VRLSYLHI | 42 |
| Nrip3 | 35 | 170 | LVITLASLRL | 180, |
| | | 194 | LSLGLQTL | 201 |
| Oct4 | 36 | 190 | LQLSFKNMCKLRPLL | 204 |
| Oligo2 | 37 | 106 | LQQLRLKI | 113 |
| Pax6 | 38 | 243 | LAAKIDL | 249 |
| Pax7 | 39 | 152 | LVSSISRVLRI | 162 |
| Pdx1 | 40 | 81 | HLHHHLPAQLALP | 93 |
| Prdm8 | 41 | 117 | LRRIAKDEELL | 127 |
| Satb2 | 42 | 621 | ISLEALGIL | 629 |
| Shox2 | 43 | 339 | IADLRL | 344 |
| Sox2 | 44 | 70 | ISKRLGAEWKLL | 82 |
| Sox9 | 45 | 134 | ELSKTGKLWRLL | 146 |
| Tbr1 | 46 | 306 | FGKLKL | 311 |
| Tbx5 | 47 | 149 | LVSFQKLKL | 157 |
| Tbx18 | 48 | 239 | VISFDKLKL | 247 |
| Zic1 | 49 | 28 | VAERDVGLGI | 37 |

### EXAMPLE 2

The following is an example of characterization of a transducible material comprising an isolated polypeptide that comprises an effector domain with a mutated NES.

In this example, transducible materials derived from transcription factor Oct4 were generated, and their abilities in regulating the target gene of Oct4 were characterized.

Isolated polypeptides of sGFP (SEQ ID NO: 104), sGFP-Oct4-11R (SEQ ID NO: 155) and sGFP-Oct4(mNES)-11R (SEQ ID NO: 141) were prepared. These proteins were expressed in E. coli in inclusion body form, which were then solubilized, refolded, and further purified. For example, the strategy for obtaining each refolded polypeptide involves 6 steps: 1) growing E. coli carrying an expression plasmid, 2) inducing the synthesis of the expressed proteins as inclusion bodies, 3) purifying the inclusion bodies with freeze-thaw and detergent washing, 4) solubilizing the inclusion bodies in a 8 M urea buffer, 5) refolding the denatured protein to its native form using a refolding process, and 6) purifying the refolded protein using sizing column chromatographic procedures to separate correctly refolded protein from its partially or totally unfolded counterparts. The protein identities were confirmed by mass spectrometry and Western blot analysis.

HepG2 cells were treated with transducible materials comprising isolated polypeptide of sGFP, sGFP-Oct4-11R or sGFP-Oct4(mNES)-11R in serum-free medium for 6 hours. RNA was extracted from each sample using RNeasy Micro Kit from Qiagen and cDNA was synthesized through reverse transcription reaction. RT-qPCRs of five Oct4 target genes (Oct4, Sox2, Nanog, Smad3 and Smad4 were performed using a pair of primers specific to each target gene and a hydrolysis probe from Roche Universal ProbeLibrary on LightCycler 480 II. The expression of each gene was normalized by the expression level of GAPDH. The relative expression level of each gene in the control condition (sGFP-treated sample) was set as 1.00.

As shown in FIG. 2, transduction of sGFP-Oct4-11R does not increase the expression of Oct4 target genes. In contrast, sGFP-Oct4(mNES)-11R significantly increase the expression level of all five Oct4 target genes being tested.

### EXAMPLE 3

The following is an example of reprogramming cell fate using a tranducible material comprising an isolated polypeptide that comprises an effector domain with a mutated NES.

In this example, three versions of transducible materials derived from MyoD (the master myogenic transcription factor) were generated and their effects on reprogramming fibroblast cells into skeletal muscle progenitor cells (SMPCs) were compared. The methods for preparing the transducible materials were generally the same as that in Example 2.

In the first generation of transducible materials, a sGFP transduction domain and a poly-arginine (11R) were fused to the N-terminus and C-terminus of MyoD through a linker (SEQ ID NO: 99) respectively to generate sGFP-MyoD-11R (SEQ ID NO: 156). As shown in FIG. 5, after transduction of 10 μg/ml sGFP-MyoD-11R, high levels of sGFP-MyoD were found in more than 90% of fibroblasts at 24 hours after exposure to sGFP-MyoD in culture. However, majority of transduced sGFP-MyoD-11R were confined in the cytoplasm with minimal nuclear occupancy. The nuclear entry of sGFP-MyoD-11R was increased by using 100 μg/ml sGFP-MyoD-11R and electroporation, which successful reprogrammed very low percentage (around 10%) of FBs to MyoD+ myoblasts. However, the harsh condition of high concentrations of polypeptide and electroporation led to death of many fibroblasts.

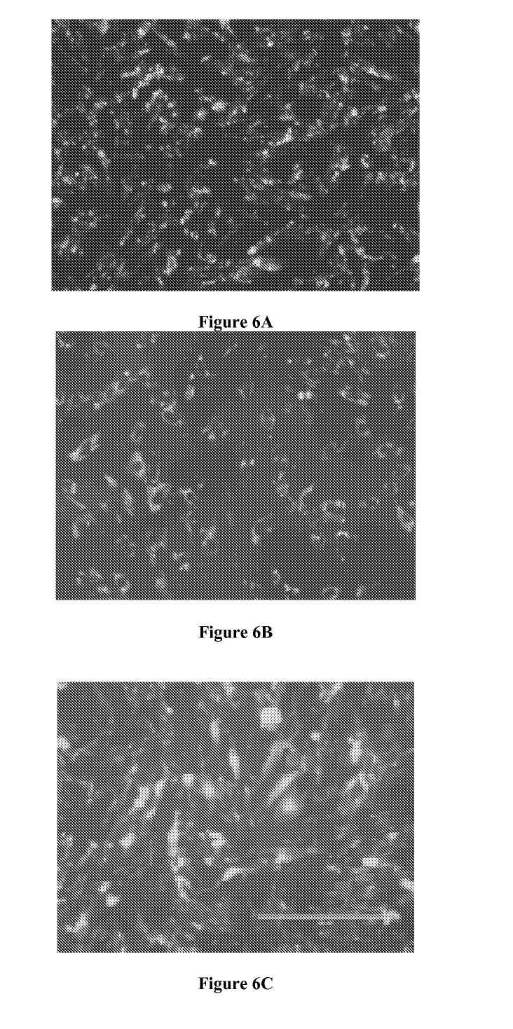

The second generation of transducible materials, a strong classic nuclear localization sequence (cNLS), the SV40 T antigen NLS, was added in-between sGFP and MyoD-11R (SEQ ID NO: 157 and FIG. 3). As shown in FIG. 6, when tranduced with 10 μg/ml sGFP-NLS-MyoD-11R, more transducible materials were observed in nuclei of fibroblasts as compared to the first generation of tranducible materials. However, the nuclear occupancy of sGFP-NLS-MyoD-11R significantly decreased within 24 hours (FIG. 6B) and therefore led to low and inconsistent reprogramming of fibroblasts to SMPCs.

Leptomycin B (leptoB), a nuclear export blocker, was added at 5 nM to adult fibroblasts to enhance nuclear occupancy of sGFP-NLS-MyoD-11R. As shown in FIG. 6C, sGFP-NLS-MyoD-11R could stay in the nucleus slightly longer than 24 hours in the continuous presence of leptoB, but reprogramming efficiency of adult fibroblasts to SMPCs was still low (around 5%), likely due to the remaining nuclear export. Higher concentrations of leptoB might have off-target effects, which limits its use in enhancing nuclear retention of specific polypeptides.

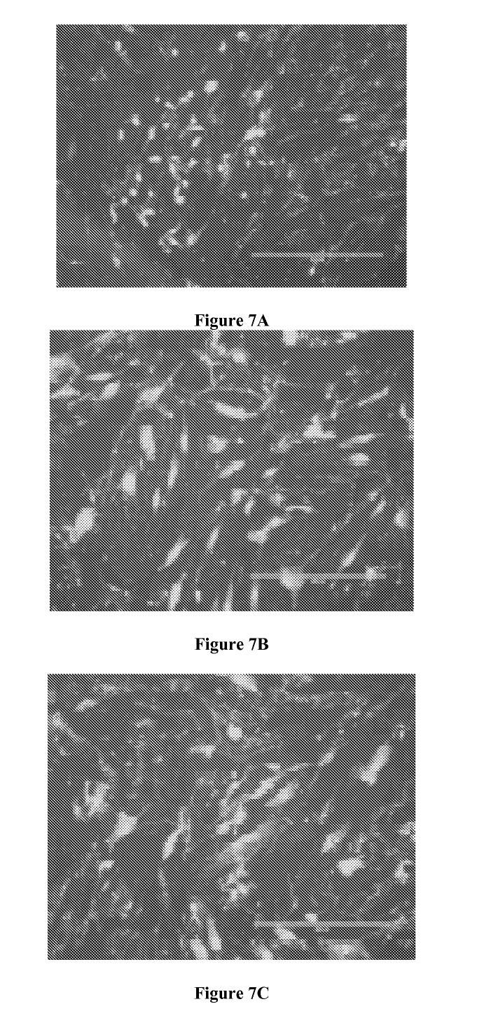

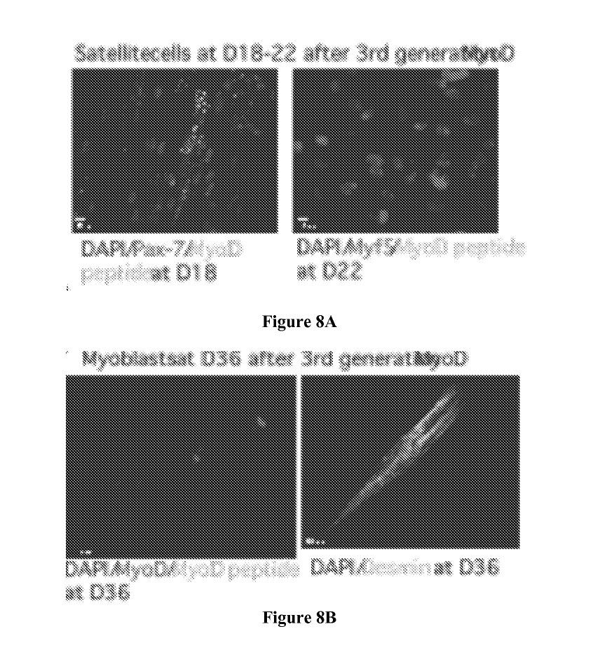

In the third generation of transducible materials, the NES of MyoD was mutated to generate sGFP-NLS-MyoD(mNES)-11R (SEQ ID NO: 127 and FIG. 3) to further improve their nuclear occupancy and transcription efficiency. As shown in FIG. 7, sGFP-cNLS-MyoD(mNES)-11R could stay in the nucleus for 48-72 hours, which successfully converted >90% of adult fibroblasts to Pax7+/MyoD− satellite cells (SCs, FIG. 8A). The results indicated that nuclear occupancy of the transducible materials for longer than 48 hours was crucial for the success of reprogramming fibroblasts. These SCs were positive for Myf5 (>90% of SCs) at 22 days after transduction (FIG. 8B). Also, these SCs could be induced to differentiate to myoblasts with downregulation of Pax7 and upregulation of MyoD via a published myogenic protocol (Cosgrove et al, Rejuvenation of the muscle stem cell population restores strength to injured aged muscles, Nat Med., 20:255-64 (2014)). More importantly, sGFP-NLS-MyoD(mNES)-11R polypeptides that entered the cells were almost completely exported from nuclei to cytoplasm at >72 hours after transduction regardless of the existence of leptomycin B. Also, fluorescent imaging of the sGFP indicated that these third generation transducible materials were fully degraded after 2-3 weeks in culture (FIG. 8A). Thus, by using sGFP-NLS-MyoD(mNES)-11R, efficient fibroblasts conversion to SMPCs can be achieved in a controlled fashion. The risk of extensive durations of MyoD activation is completely avoided. Most myogenic analysis was performed at >2 weeks after polypeptide transduction to avoid confounding issues from added exogenous polypeptides. In summary, adult fibroblast can be successfully reprogrammed to SCs using sGFP-NLS-MyoD(mNES)-11R with defined kinetics (2-day nuclear occupancy and 2-3 week peptide lifespan).

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A transducible material comprising an isolated polypeptide capable of transducing and retaining in a cell nucleus, said isolated polypeptide comprising an effector domain, wherein the effector domain comprises an amino acid sequence mutated from a nuclear export signal (NES).

2. (canceled)

3. The transducible material of claim 1, wherein the NES comprises an amino acid sequence of Y¹-X₍₂₋₃₎-Y²-X-Y³, wherein Y¹, Y² and Y³ are independently selected from the group consisting of leucine, isoleucine, valinine, phenylalanine and methionine, and X=spacing amino acid.

4. The transducible material of claim 3, wherein the NES comprises an amino acid residue sequence of LX₍₁₋₃₎LX₍₂₋₃₎LXL, wherein L=leucine and X=spacing amino acid.

5. The transducible material of claim 1, wherein the NES is mutated by replacing at least one of Y¹, Y² and Y³ with alanine.

6. The transducible material of claim 1, wherein the effector domain is derived from a protein selected from the group consisting of Ascl1, Baf60c, Bach2, Brg1, Brm, Brn2, Brn4, Chd5, cMyc, Dlx1, Dlx2, Ets2, Foxp1, Foxp2, Foxp3, Gata4, Hand1, Hes1, Hes5, Isl1, Lhx2, Lin28, Klf4 MafA, Mef2c, Mesp1, MyoD, Myt1L, Nanog, Nap1L2, NeuroD1, NeuroD2, Ngn2, Ngn3, Nhlh2, Nkx2.2, Nkx2.5, Nkx6.1, Npas4, Nrip3, Oct4, Oligo2, Pax6, Pax7, Pdx1, Prdm8, Satb2, Shox2, Sox2, Sox9, Tbr1, Tbx5, Tbx18 and Zic1.

7. The transducible material of claim 1, wherein the effector domain comprises an amino acid sequence selected from the group consisting of SEQ ID NO: 50-98.

8. The transducible material of claim 1, further comprising an agent that inhibits nuclear export.

9. The transducible material of claim 8, wherein the agent is leptomycin B.

10. The transducible material of claim 1, further comprising a nuclear localization signal (NLS).

11. The transducible material of claim 10, wherein the NLS is exogenous to the effector domain.

12. The transducible material of claim 11, wherein the NLS is linked to the effector domain covalently, non-covalently or via a linker.

13. The transducible material of claim 11, wherein the NLS is a SV40 large T antigen NLS, a c-Myc NLS or a nucleoplasmin NLS.

14. The transducible material of claim 1, further comprising a transduction domain.

15. The transducible material of claim 14, wherein the transduction domain is linked to the effector domain covalently, non-covalently or via a linker.

16. The transducible material of claim 14, wherein the transduction domain is selected from the group consisting of a protein transduction domain, a cell penetrating peptide, a cell permeating peptide, an activatable cell penetrating peptide, a cell-targeting peptide and a polymer.

17. The transducible material of claim 16 wherein the protein transduction domain is a supercharged protein.

18. The transducible material of claim 1, wherein the effector domain is inherently transducible.

19. The transducible material of claim 14, wherein the isolated polypeptide comprises an amino acid sequence of SEQ ID NO: 106-154.

20. A composition comprising a biological sample and the transducible material of claim 1, wherein the transducible material has transduced into the biological sample.

21. The composition of claim 20, wherein the biological sample is a cell, a tissue, or an organ from a biological organism.

22. A method for reprogramming a biological sample, comprising: exposing the biological sample to the transducible material of claim 1.

23. The method of claim 22, wherein the biological sample is reprogrammed so as to increase or decrease proliferation, change cell transcription network or metabolism, or to induce differentiation, transdifferentiation, retrodifferentiation, transdetermination, dedifferentiation, apoptosis or morphogenesis.

24. The method of claim 22, wherein the biological sample is a cell, wherein the cell is reprogrammed to change from a first type cell to a second type cell.

25. The method of claim 24, wherein the first type cell is a fibroblast and the second type cell is a muscle progenitor cell.

26-30. (canceled)
